(19) United States
(12) Patent Application Publication
(10) Pub. No.: US 2024/0060977 A1
(43) Pub. Date: Feb. 22, 2024

(54) USE OF BIOMARKERS FOR PREDICTING THE CLINICAL AND/OR TREATMENT OUTCOME OF A HUMAN SUBJECT

(71) Applicant: ALPSPITZ BIOSCIENCE GMBH, Gattendorf (DE)

(72) Inventor: Michael KRETZER, Hof (DE)

(21) Appl. No.: 18/271,422

(22) PCT Filed: Jan. 10, 2022
(86) PCT No.: PCT/EP2022/050348

§ 371 Date: Jul. 7, 2023

(30) Foreign Application Priority Data
Jan. 8, 2021 (DE) 10 2021 100 237.0
Jan. 8, 2021 (EP) 21150746.2

(51) Int. Cl. G01N 33/566

(52) U.S. Cl. CPC G01N 33/566; G01N 2333/5412; G01N 2800/52

(57) ABSTRACT

Disclosed is a method of calculating a predictive score and for treating a human subject based on at least two biomarkers in the saliva of human subjects. The predictive score of the invention is used to predict the risk of an adverse clinical and/or treatment outcome in a human subject at risk of a clinical condition.

The present invention pertains to the use of biomarkers for predicting the clinical and/or treatment outcome of a human subject. More specifically, the present invention pertains to the use of at least two salivary biomarkers for predicting clinical and/or treatment outcome.

### BACKGROUND OF THE INVENTION

Biomarkers are critical to the rational development of medical therapeutics and patient stratification. A number of subtypes of biomarkers have been defined according to their putative applications. A single biomarker may meet multiple criteria for different uses, however, it is decisive to develop evidence for each definition. Generally, biomarkers can be used as diagnostic biomarkers, pharmacodynamic/response biomarkers, or predictive biomarkers.

Thus far, various biomarkers have been used in predicting or prognosing treatment response, course of the disease, or response to a certain treatment regimen: For example, EP2554994 B1 discloses the use of soluble interleukin-6 receptor as a biomarker for predicting response to chemoradiotherapy for a squamous cell carcinoma. WO 2013/131093 A1 discloses the use of at least 3 cytokines of a panel of urinary cytokines taken prior to and following Bacillus Calmette Guerin (BCG) therapy to predict the risk of relapse in a cancer patient. WO02/099114 A1 discloses the use of TGFb1, IGFBP-3, IL6, IL-6sR, uPA, uPAR in a blood plasma samples as biomarkers to determine the prognosis of a patient following local bladder cancer therapy.

Other strategies to predict treatment outcome have employed the ratio of prognostic RNA transcripts to predict treatment outcome in malignant melanoma (e.g. WO 2011/000388 A1), or to predict patient response to a treatment with an anti-EGFR antibody (WO2010/145796 A1).

Blood cytokine levels of different panels of cytokines have been suggested as prognostic and diagnostic biomarkers in multiple diseases such as rheumatoid arthritis (see e.g. Mediators Inflamm. 2014; 2014: 545493), Huntington's disease (Journal of Huntington's Disease 7 (2018) 109-135), or chronic obstructive pulmonary disease (COPD) (Respiratory Research (2017) 18:180), or recently blood IL-6 levels have been implicated in the disease severity of COVID-19 patients (Cytokine and Growth Factor Reviews 53 (2020) 13-24).

Biomarkers and/or biomarker panels used in prior art to predict e.g. treatment response or prognose disease severity for a given patient comprise, however, a disease-specific panel of biomarkers on protein and transcript level. Additionally, most tests are based on the analysis of blood samples which require appropriate handling of the samples and work-up of the samples for subsequent biomarker analysis.

Thus, there is a need for a reliable method of predicting the clinical and/or treatment outcome for patients that can be used for a broad range of clinical conditions or treatment regimens and which does not require the handling of blood samples.

### SUMMARY OF THE INVENTION

The present invention addresses the need to provide a method for predicting the clinical and/or treatment outcome by providing a method to calculate a predictive score (S_{P}) based on two salivary biomarkers according to a first embodiment, which provides for a method of predicting the clinical and/or treatment outcome in a human subject at risk of prospective clinical condition comprising
(i) determining the salivary concentration of Interleukin-6 (IL-6) in a salivary sample of said human subject; and
(ii) determining the concentration of salivary neutrophils in said salivary sample of said human subject.

According to one embodiment, the method of the present invention indicates that a human subject is expected to have a higher risk of prospective decrease of organ functions and a higher risk of medical complications for every unit of increase of the salivary concentration of IL-6 said; and for every unit of increase of the concentration of salivary neutrophils said human subject is expected to have a higher risk of said clinical condition and a higher risk of medical complications.

According to one embodiment, the present invention provides for a predictive score which is calculated using a Theta Heaviside function using at least a normalized salivary IL-6 value (Ñ_{IL-6}) and a normalized salivary neutrophil value (Ñ_{NP}), wherein the normalized salivary IL-6 value (Ñ_{IL-6}) and normalized salivary value (Ñ_{NP}) are determined according to a scoring algorithm, comprising the steps of
a) assigning a predictive IL-6 score Ñ_{IL-6}=0, if the salivary IL-6 concentration is lower than or equal to the normalization value of IL-6,
b) calculating Ñ_{IL-6} as the ratio of salivary IL-6 concentration:IL-6 normalization value and wherein the numerical value of 1 is subtracted from the normalization result yielding Ñ_{IL-6}, for a salivary IL-6 concentration which is greater than the IL-6 normalization value,
c) assigning a predictive neutrophil (NP) score Ñ_{NP}=0, if the salivary neutrophil concentration is lower than or equal to the normalization value for salivary neutrophils,
d) calculating Ñ_{NP} as the ratio of salivary neutrophil concentration: salivary neutrophil normalization value and wherein the numerical value of 1 is subtracted from the normalization result yielding Ñ_{NP}, for a salivary neutrophil concentration which is greater than the IL-6 normalization value, and wherein the predictive score (S_{P}) is calculated as S_{P}=Ñ_{NP}+Ñ_{IL-6}.

According to one embodiment, the normalization values of salivary IL-6 and salivary neutrophils of the inventive method to calculate the predictive score are 10 pg/ml salivary IL-6 and the normalization value for salivary neutrophils is 90 cells/μl.

According to some embodiments, a predictive score (S_{P}) according to the invention of S_{P}=0 indicates a low risk of an adverse clinical and/or treatment outcome and a predictive score according to the invention of S_{P}>0 indicates an increased risk of an adverse clinical and/or treatment outcome.

According to one embodiment, the inventive predictive score is used to determine the clinical and/or treatment outcome between two or more human subjects at risk of prospective clinical condition, wherein the method comprises calculating a predictive score S_{P} for each human subject, wherein the subject with the lowest predictive score has the lowest risk of an adverse clinical and/or treatment outcome.

According to one embodiment, the predictive score according to the invention as disclosed above is calculated using one or more additional biomarkers in addition to the concentration of salivary IL-6 and salivary neutrophils, wherein the predictive score S_{P} is calculated as

S_{P}=Ñ_{NP}+Ñ_{IL-6}+Σᵢ₌₁ⁿÑ(i) (I)
wherein Ñ(i), is the predictive score of the one or more additional biomarker, wherein n is from 1 to 25, depending on the number of additional biomarkers employed to calculate the predictive score. According to the invention, the normalized value Ñ(i) is determined according to a scoring algorithm which comprises the steps of
(i) assigning the numerical value of zero (0) to Ñ(i) if the concentration of said biomarker in said salivary sample is lower than or equal to the normalization value of said biomarker;
(ii) in case the concentration of said biomarker in a respective sample is greater than the normalization value for said biomarker, Ñ(i) is calculated as the ratio of said salivary biomarker (i) concentration: biomarker (i) normalization value, whereby the numerical value of 1 is subtracted from the normalization result yielding Ñ(i) for said salivary biomarker(i).

According to one embodiment, the one or more additional biomarkers according to the invention which can be used for the calculation of the predictive score of the invention is selected from the group comprising interleukin receptor antagonist, interleukin-1 beta, interleukin-7, interleukin-8, interleukin-10, interleukin-13, colony stimulating factor 3, C-X-C motif chemokine ligand 10, C-C motif chemokine ligand 3, C-C motif chemokine ligand 11, interferon-gamma, tumor necrosis factor-alpha, alpha-synuclein, amyloid beta 1-42, protein t-TAU, matrix metalloproteinase-8, tumor markers CA125, TPS, CA19-9, CEA, CA 15-3, SCC.

In one embodiment, the clinical condition according to the invention is a decrease in organ function and/or a medical complication, wherein the decrease in organ function is one of lung function, heart function, renal function, cardiovascular function, musculoskeletal function, endocrine function, gastrointestinal function, or neurological function.

In one embodiment, the medical complication is one of renal failure, increased frailty according to the CSHA Frailty Index, thromboembolic complications, gastrointestinal complications, cardiac complications, neurologic complications, metabolic complications.

According to one embodiment, the prospective decrease in organ function and/or the risk of medical complications is related to or a function of age of the human subject, or related to inflammatory disease, degenerative syndromes, malignant tumors, or viral infections of the human subject.

According to one embodiment, prospective decrease in organ function and/or the risk of medical complications is caused by viral infection, such as e.g. infection with a virus of the family of coronaviridae, such as SARS-CoV, or SARS-CoV2.

In some embodiments, the calculation of the predictive score S_{P} according to the invention is done using a device carrying out point-of-care testing.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention. The word “substantially” does not exclude “completely” e.g., a composition which is “substantially free” from Y may be completely free from Y. Where necessary, the word “substantially” may be omitted from the definition of the invention.

The term “about” in relation to a numerical value x as used herein means x±10%.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will supersede any other definition. In addition, the materials, methods, and examples are illustrative only and not intended to be limiting.

In a first aspect, the present invention provides for a method of predicting the clinical and/or treatment outcome in a human subject at risk of prospective clinical condition comprising
(i) determining the salivary concentration of Interleukin-6 (IL-6) in a salivary sample of said human subject; and
(ii) determining the concentration of salivary neutrophils in said salivary sample of said human subject.

It was surprisingly found that determining the at least two salivary biomarkers the inventive method can be used to predict the clinical and/or treatment outcome in a human subject at risk of prospective clinical condition.

According to one embodiment of the invention, it was found that for every unit increase of the salivary concentration of IL-6 in a human subject correlates with a higher risk of a prospective decrease in organ function and that for every unit increase of the salivary concentration of neutrophils said human subject is has a higher risk of said clinical condition and a higher risk of medical complications. The term “IL-6” as used in the present invention refers to the cytokine interleukin-6. IL-6 is a pro-inflammatory cytokine which in humans is encoded by the IL6 gene. IL-6 binds to the IL-6 receptor (IL6R) forming a complex which then associates to the signaling subunit IL6ST/gp130 to trigger the intracellular IL6-signaling pathway. The interaction with the membrane-bound IL6R and IL6ST stimulates ‘classic signaling’, whereas the binding of IL-6 and soluble IL6R to IL6ST stimulates ‘trans-signaling’. Alternatively, ‘cluster signaling’ occurs when membrane-bound IL6:IL6R complexes on transmitter cells activate IL6ST receptors on neighboring receiver cells. IL-6 has pleotropic functions ranging from hematopoiesis and metabolic regulation to inflammation, autoimmunity and acute phase response. IL-6 modulates host defense through a number of immune stimulating mechanisms: control of monocytes and their differentiation into macrophages, modulation of antigen-dependent B cell differentiation, increased IgG production by B cells, and promotion of Th2 response by inhibiting Th1 polarization.

In addition IL-6 has been implicated in the differentiation of CD4+ T cell subsets. The term “salivary IL-6” or “salivary interleukin-6” both terms of which may be used interchangeable throughout this application refers to IL-6 as comprised in human saliva. The IL-6 concentration in a saliva sample may e.g. be determined using commercially available ELISA kits. The term “unit” as used herein refers to the concentration of IL-6 in a given saliva sample in pg/ml, or pg/μl saliva. The term unit as used according to the present invention may e.g. also be used to refer to a normalized value of IL-6, e.g. the salivary concentration as determined by a commercial IL-6 ELISA may by normalized by a reference value which may be the average salivary IL-6 concentration of a control group or reference group.

According to one embodiment, the salivary samples which are used in the inventive method may be collected prior to and during a known clinical disease. Saliva composition may vary over the course of a day, because of which it may be advantageous to collect saliva samples at or at about the same time of day. Other factors that may impact saliva composition and or the amount of saliva secreted should be avoided prior to saliva sample collection in addition to collecting the saliva at the same time of day. For example, foods with high sugar or acidity, or high caffeine content, immediately before sample collection should thus be avoided. In addition, such foods or beverages may compromise the respective IL-6 assay by lowering saliva pH and increasing bacterial growth. Saliva samples of the invention may e.g. be collected by oral swaps, or passive drool. The use of passive drool samples is both cost effective and approved for use with a large number of all analytes, in addition sample collection by passive drool maintains sample integrity. It is thus is preferred, that passive drool is used for the collection of saliva samples according to the invention. Passive drool samples may e.g. be collected into sealable and sterile polypropylene vials. The collection of a saliva sample may be done from about 1, 2, 3, 4, 5, 6, 7, 8, 9, 10 days to about 12, 14, 16, 18, 20, 21, 28, 35 days, or from about 21, 28, 35 days to about 40, 50, 60, 90, 120, 180 days, or about 7, 8, 9, 10, 11, 12 months prior to a clinical condition. The collection of the saliva sample may e.g. be done as part of a routine checkup prior to manifestation of a clinical disease. According to one embodiment, the saliva sample may e.g. be obtained during the clinical disease. Accordingly, the saliva sample may be taken at any time during the course of the clinical disease. In some embodiments, more than one saliva sample e.g. 2, 3, 4, 5, 6, 7, 8, 9, or 10 saliva samples may be collected e.g. by passive drool, during the course of the clinical disease to monitor the course of the disease using the inventive method. The analysis of multiple saliva samples using the inventive method may e.g. be advantageous to monitor a decrease in organ function during the course of the clinical disease, or predict medical complications using the inventive method.

The term “salivary neutrophils” as used according to the invention refers to neutrophils which are present in the human saliva. Neutrophils, which may e.g. also be referred to as neutrocytes or heterophils, are the most abundant type of granulocytes and make up 40% to 70% of all white blood cells in humans and form an essential part of the innate immune system. Neutrophils can be identified by CD11b, CD16, and CD66b expression as markers that are consistently expressed on neutrophils independent of the cell location, level of activation and disease state (see e.g. Exp Cell Res. 2016 Mar. 15; 342(2):200-9). Alternatively, in humans, neutrophils may e.g. be distinguished from eosinophils and monocytes based on the expression of both CD15 and CD16/Fc gamma RIII, concomitant with the lack of expression of CD14.

According to one embodiment, the concentration of salivary neutrophils in the inventive method as disclosed herein can e.g. be determined by determining the concentration and/or the activity of salivary leukocyte esterase (LE) which is released by salivary neutrophils.

Accordingly, the method of the invention comprises the step of determining the concentration and/or activity of salivary neutrophils by determining the salivary concentration of leukocyte esterase and correlating the concentration of LE to the concentration of salivary neutrophils in the saliva sample from said human subject. LE concentration in a saliva sample can e.g. be determined by using commercially available quantitative sandwich LE ELISA kits according to the manufacturer's instructions provided with the respective assay. Alternatively, assays can be used which provide the enzymatic activity in a given sample which is correlated to the number of salivary neutrophils in the sample, which can be determined using commercially available LE reagent strips which allow quantitative determination of LE concentration and/or activity. For example, LE activity can be determined in one or more control saliva samples from individuals for which the concentration of salivary neutrophils has been determined by FACS analysis (CD15-positive, CD16/Fc gamma RIII-positive cells, which lack of expression of CD14), or e.g. by the method as disclosed in Ashkenazi and Dennison J. Dent. Res. 1989 68: 1256 and correlated to the number of salivary neutrophils in said sample or samples. Accordingly, LE concentrations, or LE enzymatic activities above or below the concentration/activity in the control group indicate a greater or reduced number of salivary leukocytes in the sample compared to the control sample. It may e.g. be advantageous to establish a standard curve for the correlation of salivary neutrophils to LE concentration (or LE activity/sample volume) using samples with a pre-determined number of salivary neutrophils for correlating LE concentration/activity to the number of salivary neutrophils which can then be used in the inventive method to calculate the predictive score (S_{P}).

The term “clinical condition” as used according to the present invention refers to a medical complication, e.g. an unfavorable evolution of a disease, health condition or treatment. Medical complications may e.g. adversely affect prognosis, or outcome of a disease. Complications may generally involve a worsening in severity of disease or the development of new signs, symptoms, or pathological changes which may become widespread throughout the body and affect other organ systems of a human subject. Complications may e.g. lead to the development of new diseases resulting from a previously existing disease. Complications may also arise as a result of various treatments due to e.g. adverse effects of each of the various treatment regimens. The development of complications depends on a number of factors, such as e.g. the degree of vulnerability, susceptibility, age, health status, and immune system condition.

In one embodiment, the present invention provides for an algorithm to calculate a predictive score which is based on the salivary concentration of IL-6 and the concentration of salivary neutrophils, whereby a Theta Heaviside function is used on a normalized salivary IL-6 value (Ñ_{IL-6}) and on a normalized salivary neutrophil value (Ñ_{NP}). According to the invention, the normalized salivary IL-6 value (Ñ_{IL-6}) and normalized salivary value (Ñ_{NP}) are determined according to a scoring algorithm which comprises the steps of
i) assigning a predictive IL-6 score Ñ_{IL-6}=0, if the salivary IL-6 concentration is lower than or equal to the normalization value of IL-6,
ii) calculating Ñ_{IL}-6 as the ratio of salivary IL-6 concentration:IL-6 normalization value and wherein the numerical value of 1 is subtracted from the normalization result yielding Ñ_{IL}-6, for a salivary IL-6 concentration which is greater than the IL-6 normalization value,
iii) assigning a predictive neutrophil (NP) score Ñ_{NP}=0, if the salivary neutrophil concentration is lower than or equal to the normalization value for salivary neutrophils,
iv) calculating Ñ_{NP} as the ratio of salivary neutrophil concentration: salivary neutrophil normalization value and wherein the numerical value of 1 is subtracted from the normalization result yielding Ñ_{NP}, for a salivary neutrophil concentration which is greater than the IL-6 normalization value, and wherein the predictive score of the invention (S_{P}) is calculated as S_{P}=Ñ_{NP}+Ñ_{IL-6}.

The term “Theta Heaviside function” as used herein refers to the Heaviside step function. The term “normalization value” as used according to the invention refers to the concentration of salivary IL-6 and salivary neutrophils in a reference group wherein the reference group for salivary IL-6 and salivary neutrophils may be identical, or different reference groups.

According to the invention the salivary normalization value for IL-6 is 10 pg/ml (see e.g. Paneer Selvam N et al, Salivary interleukin-6. Asia-Pac. J. Clin. Oncol. 11: 236-241, 2015) and the salivary normalization value for neutrophils is 90 neutrophils/μl (Domnich M et al, Oral Neutrophils. Front. Immunol. 11: 565683, 2020). Depending on the reference groups chosen, these values may vary without affecting the prognostic value of the predictive score.

According to one embodiment, a predictive score (S_{P}) of S_{P}=0 indicates a low risk of an adverse clinical and/or treatment outcome and a predictive score S_{P}>0 indicates an increased risk of an adverse clinical and/or treatment outcome. Accordingly, a human subject with a predictive score S_{P}=0 according to the invention has a low risk of being afflicted with an adverse clinical and/or treatment outcome. The term “low risk” as used herein refers to a probability of less than 50%, preferably of less than 40%, 30%, 25%, 12.5%, 10%, 5%, 2.5% of an occurrence of an event such as an adverse clinical and/or treatment outcome. For example, the term “low risk” according to the invention may be used in comparison to a control group for any given event, such as an adverse clinical and/or treatment outcome whereby the probably of the event of not occurring may be expressed as

$$P ’ = ( 1 - \frac{number of favorable outcomes}{total number of outcomes} ) ⋆ 100 \%$$

For example, the probability P′ of an adverse clinical and/or treatment outcome may be calculated for a control group for e.g. the probability of organ failure, or metabolic complications. In case the predictive score S_{P} of a human subject is zero, this indicates that the probability of the adverse clinical and/or treatment outcome to occur with the human subject is smaller than for the control group and accordingly human subject is assigned a “low risk” according to the invention.

The term “at risk” according to the invention refers to probability of the occurrence of an adverse event which is greater than that of a control group. For example, a human subject at risk of a prospective clinical condition may have a probability of occurrence (P_{c}) that is greater than the average probability of a control group, which may be expressed as:

$${P}_{C} > P = \frac{number of favorable outcomes}{total number of outcomes}$$

According to one embodiment, a human subject is assigned an “increased risk” if the predictive score S_{P}>0 indicating that for the human subject the likelihood of occurrence of the adverse clinical and/or treatment outcome is greater than that of the control group.

Accordingly, a human subject is assigned a “decreased” or “lower” risk if e.g. for the respective human subject the likelihood of occurrence of the adverse clinical and/or treatment outcome is lower than that of the control group or a group of human subjects said human subject is compared to (e.g. Pc<P, wherein P is one of the average probability of a control group, or group or individuals it is compared to).

According to one embodiment, the inventive predictive score is used to determine the clinical and/or treatment outcome between two or more, e.g. 2, 3, 4, 5, 6, 7, 8, 9, 10, 25, 50, 75, 100, 1000 or more human subjects at risk of a prospective clinical condition. The inventive method comprises calculating a predictive score S_{P} for each human subject (S_{Pi}), comparing the resulting predictive scores, whereby the subject with the lowest predictive score has the lowest risk of an adverse clinical and/or treatment outcome, whereby the According to one embodiment, the predictive score of the invention is e.g. calculated using one or more, e.g. 1, 2, 3, 4, 5, 6, 7, 8, 9, 10, 11, 12, 13, 14, 15, 16, 17, 18, 19, 20, 21, 22, 23, 24 or 25 additional biomarkers, wherein the predictive score S_{P} is calculated as

S_{P}=Ñ_{NP}+Ñ_{IL-6}+Σᵢ₌₁ⁿÑ(i),

wherein Ñ(i), is the predictive score of the one or more additional biomarker (i) as disclosed herein and wherein n corresponds to the number of additional biomarkers that are used to calculate the predictive score according to the invention. For example, n may be 1, 2, or 3 if one, two, or three additional biomarkers in addition to the salivary concentration of IL6 and salivary neutrophils are used. The normalized value (Ñ(i)) of a respective additional biomarker as disclosed herein is determined according to a scoring algorithm which comprises the steps of
assigning the numerical value of zero (0) to Ñ(i) (e.g. Ñ(i)=0), if the concentration of said of said one or more biomarker in said salivary sample is lower than or equal to the normalization value of said one or more biomarker;
if the concentration of said one or more salivary biomarker(i) is greater than the respective normalization value of said biomarker (i), Ñ(i) is calculated as the ratio of said salivary biomarker (i) concentration:biomarker(i) normalization value and wherein the numerical value of 1 is subtracted from the normalization result yielding Ñ(i):

$$\tilde{N} ( i ) = \frac{concentration salivary biomarker ( i )}{normailzation value biomarker ( i )} - 1$$

According to a preferred embodiment, the one or more additional biomarkers of the invention which may be used to calculate the predictive score according to the invention are selected from the group comprising the one or more additional biomarker is selected from the group comprising interleukin receptor antagonist, interleukin-1 beta, interleukin-7, interleukin-8, interleukin-10, interleukin-13, colony stimulating factor 3, C-X-C motif chemokine ligand 10 (CXCL10), C-C motif chemokine ligand 3, C-C motif chemokine ligand 11, interferon-gamma, tumor necrosis factor-alpha, alpha-synuclein, amyloid beta 1-42, protein t-TAU, matrix metalloproteinase-8, CA125 (cancer antigen 125, MU16), TPS, CA19-9 (sialyl-Lewis^{a}, sLe^{a}), CEA (carcinoembryonic antigen, CEACAM), CA 15-3, or SCC. Accordingly, the predictive score (S_{P}) according to the invention may e.g. be calculated using one, two, three, four, five, six, seven, eight, nine, ten, or more additional biomarkers as disclosed hereinabove. For example, any combination of the biomarkers as disclosed above may be used for calculating the predictive score of the invention. For example, the predictive score S_{P} according to the invention may be calculated as: Ñ_{NP}+Ñ_{IL-6}+Ñ_{(interleukin receptor antagonist)}, Ñ_{NP}+Ñ_{IL-6}+Ñ_{(interleukin-1 beta)}, Ñ_{NP}+Ñ_{IL-6}+Ñ₍ᵢₙₜₑᵣₗₑᵤₖᵢₙ₋₇₎, Ñ_{NP}+Ñ_{IL-6}+Ñ₍ᵢₙₜₑᵣₗₑᵤₖᵢₙ₋₈₎, Ñ_{NP}+Ñ_{IL-6}+Ñ₍ᵢₙₜₑᵣₗₑᵤₖᵢₙ₋₁₀₎, Ñ_{NP}+Ñ_{IL-6}+₍ᵢₙₜₑᵣₗₑᵤₖᵢₙ₋₁₃₎, Ñ_{NP}+Ñ_{IL-6}+Ñ_{(colony stimulating factor 3)}, Ñ_{NP}+Ñ_{IL-6}+Ñ_{(C-X-C motif chemokine ligand 10)}, Ñ_{NP}+Ñ_{IL-6}+Ñ_{(C-C motif chemokine ligand 3)}, Ñ_{NP}+Ñ_{IL-6}+Ñ_{(C-C motif chemokine ligand 1)}, Ñ_{NP}+Ñ_{IL-6}+Ñ_{(interferon-gamma)}, Ñ_{NP}+Ñ_{IL-6}+Ñ_{(tumor necrosis factor-alpha)}, Ñ_{NP}+Ñ_{IL-6}+Ñ_{(alpha-synuclein)}, Ñ_{NP}+Ñ_{IL-6}+Ñ_{(amyloid beta 1-42)}, Ñ_{NP}+Ñ_{IL-6}+Ñ_{(protein t-TAU)}, Ñ_{NP}+Ñ_{IL-6}+Ñ_{(matrix metalloproteinase-8)}, Ñ_{NP}+Ñ_{IL-6}+Ñ_{(CA125)}, Ñ_{NP}+Ñ_{IL-6}+Ñ_{(TPS)}, Ñ_{NP}+Ñ_{IL-6}+Ñ_{(CA19-9)}, Ñ_{NP}+Ñ_{IL-6}+Ñ_{(CEA)}, Ñ_{NP}+Ñ_{IL-6}+Ñ_{(CA15-3)}, Ñ_{NP}+Ñ_{IL-6}+Ñ_{(SCC)}, or e.g. Ñ_{NP}+Ñ_{IL-6}+Ñ_{(amyloid beta 1-42)}+Ñ_{(protein t-TAU)}, Ñ_{NP}+Ñ_{IL-6}+Ñ_{(interleukin receptor antagonist)}+Ñ_{(interleukin-1 beta)}, Ñ_{NP}+Ñ_{IL-6}+Ñ_{(interleukin receptor antagonist)}+Ñ_{(interferon-gamma)}, Ñ_{NP}+Ñ_{IL-6}+Ñ_{(colony stimulating factor 3)}+Ñ_{(interleukin-1 beta)}, Ñ_{NP}+Ñ_{IL-6}+Ñ_{(CEA)}+Ñ_{(tumor necrosis factor-alpha)}, Ñ_{NP}+Ñ_{IL-6}+Ñ_{(CEA)}+Ñ_{(interferon gamma)}, Ñ_{NP}+Ñ_{IL-6}+Ñ_{(matrix metalloproteinase-8)}+Ñ_{(tumor necrosis factor-alpha)}, Ñ_{NP}+Ñ_{IL-6}+Ñ_{(CEA)}+Ñ_{(SCC)}, Ñ_{NP}+Ñ_{IL- 6}+Ñ_{(CA125)}+Ñ_{(SCC)}, Ñ_{NP}+Ñ_{IL-6}+Ñ_{(CA125)}+Ñ_{(matrix metalloproteinase-8)}, Ñ_{IL-6}+Ñ_{(CA125)}+Ñ_{(interferon-gamma)}, Ñ_{IL-6}+Ñ_{(CA125)}+Ñ_{(tumor necrosis factor-alpha)}, Ñ_{IL-6}+Ñ_{(CA125)}+Ñ_{(interleukin receptor antagonist)}, Ñ_{IL-6}+Ñ_{(CA125)}+Ñ_{(CEA)}, Ñ_{IL-6}+Ñ_{(CA125)}+Ñ_{(CA19-9)}, Ñ_{IL-6}+Ñ_{(CA125)}+Ñ_{(CA15-3)}, Ñ_{NP}+Ñ_{IL-6}+Ñ_{(interferon gamma)}+Ñ_{(SCC)}, Ñ_{NP}+Ñ_{IL-6}+Ñ_{(interferon gamma)}+Ñ_{(matrix metalloproteinase-8)}, Ñ_{NP}+Ñ_{IL-6}+Ñ_{(C-C motif chemokine ligand 1)}+Ñ_{(C-X-C motif chemokine ligand 10)}, Ñ_{NP}+Ñ_{IL- 6}+Ñ_{(C-C motif chemokine ligand 1)}+Ñ₍ᵢₙₜₑᵣₗₑᵤₖᵢₙ₋₈₎, Ñ_{NP}+Ñ_{IL-6}+Ñ_{(C-C motif chemokine ligand 1)}+Ñ₍ᵢₙₜₑᵣₗₑᵤₖᵢₙ₋₁₃₎, Ñ_{NP}+Ñ_{IL-6}+Ñ_{(C-C motif chemokine ligand 1)}+Ñ_{(interleukin-1 beta)}, Ñ_{NP}+Ñ_{IL-6}+Ñ_{(matrix metalloproteinase-8)}+Ñ_{(interleukin- 8)}, Ñ_{NP}+Ñ_{IL-6}+Ñ_{(matrix metalloproteinase-8)}+Ñ₍ᵢₙₜₑᵣₗₑᵤₖᵢₙ₋₇₎, Ñ_{NP}+Ñ_{IL-6}+Ñ_{(matrix metalloproteinase-8)}+Ñ₍ᵢₙₜₑᵣₗₑᵤₖᵢₙ₋₁₀₎, Ñ_{NP}+Ñ_{IL-6}+Ñ_{(matrix metalloproteinase-8)}+Ñ₍ᵢₙₜₑᵣₗₑᵤₖᵢₙ₋₁₃₎, Ñ_{NP}+Ñ_{IL-6}+Ñ_{(matrix metalloproteinase-8)}+Ñ_{(TPS)}, Ñ_{NP}+Ñ_{IL-6}+Ñ_{(matrix metalloproteinase-8)}+Ñ_{(CA19-9)}, Ñ_{NP}+Ñ_{IL-6}+Ñ_{(matrix metalloproteinase-8)}+Ñ_{(CA15-3)}, Ñ_{NP}+Ñ_{IL-6}+Ñ_{(CEA)}+Ñ_{(tumor necrosis factor-alpha)}, or e.g. Ñ_{NP}+Ñ_{IL-6}+Ñ_{(SCC)}+Ñ₍ᵢₙₜₑᵣₗₑᵤₖᵢₙ₋₁₀₎+Ñ_{(C-X-C motif chemokine ligand 10)}, Ñ_{NP}+Ñ_{IL-6}+Ñ_{(CEA)}+_{(tumor necrosis factor-alpha)}+Ñ_{(SCC)}, Ñ_{NP}+Ñ_{IL-6}+Ñ_{(amyloid beta 1-42)}+Ñ_{(protein t-TAU)}+Ñ_{(alpha-synuclein)}, Ñ_{NP}+Ñ_{IL-6}+Ñ_{(matrix metalloproteinase-8)}+Ñ₍ᵢₙₜₑᵣₗₑᵤₖᵢₙ₋₇₎+)+Ñ_{(SCC)}, Ñ_{NP}+Ñ_{IL-6}+Ñ_{(matrix metalloproteinase-8)}+Ñ_{(SCC)}+)+Ñ_{(CEA)}, Ñ_{NP}+Ñ_{IL-6}+Ñ_{(matrix metalloproteinase-8)}+Ñ_{(SCC)}+)+Ñ_{(CA19-9)}, Ñ_{NP}+Ñ_{IL-6}+Ñ_{(matrix metalloproteinase-8)}+Ñ_{(SCC)}+)+Ñ_{(CA15-3)}, Ñ_{NP}+Ñ_{IL-6}+Ñ_{(matrix metalloproteinase-8)}+Ñ_{(SCC)}+)+Ñ_{(interferon gamma)}, Ñ_{NP}+Ñ_{IL-6}+Ñ_{(CEA)}+Ñ_{(SCC)}++Ñ_{(interferon gamma)}, Ñ_{NP}+Ñ_{IL-6}+Ñ_{(CA19-9)}+Ñ_{(SCC)}+)+Ñ_{(interferon gamma)}, Ñ_{NP}+Ñ_{IL-6}+Ñ_{(CA15-3)}+Ñ_{(SCC)}+)+Ñ_{(interferon gamma)}, Ñ_{NP}+Ñ_{IL-6}+Ñ_{(CA15-3)}+Ñ_{(SCC)+)}+Ñ_{(tumor necrosis factor alpha)}, Ñ_{NP}+Ñ_{IL-6}+Ñ_{(CA125)}+Ñ_{(SCC)}+)+Ñ_{(tumor necrosis factor alpha)}, Ñ_{NP}+Ñ_{IL-6}+Ñ_{(CA125)}+Ñ_{(SCC)}+Ñ_{(CEA)}, Ñ_{NP}+Ñ_{IL-6}+Ñ_{(interleukin-1 beta)}+Ñ₍ᵢₙₜₑᵣₗₑᵤₖᵢₙ₋₇₎+Ñ₍ᵢₙₜₑᵣₗₑᵤₖᵢₙ₋₈₎, Ñ_{NP}+Ñ_{IL-6}+Ñ_{(interleukin-1 beta)}+Ñ₍ᵢₙₜₑᵣₗₑᵤₖᵢₙ₋₇₎+Ñ₍ᵢₙₜₑᵣₗₑᵤₖᵢₙ₋₁₀₎, Ñ_{NP}+Ñ_{IL-6}+Ñ_{(interleukin-1 beta)}+Ñ₍ᵢₙₜₑᵣₗₑᵤₖᵢₙ₋₇₎+Ñ₍ᵢₙₜₑᵣₗₑᵤₖᵢₙ₋₁₃₎, or e.g. Ñ_{NP}+Ñ_{IL-6}+Ñ_{(matrix metalloproteinase-8)}+Ñ_{(SCC)}+)+Ñ_{(CA15-3)}+Ñ_{(CA19-9)}, Ñ_{NP}+Ñ_{IL-6}+Ñ_{(matrix metalloproteinase-8)}+Ñ_{(SCC)}+)+Ñ_{(CA15-3)}+Ñ_{(CEA)}, Ñ_{NP}+Ñ_{IL-6}+Ñ_{(matrix metalloproteinase-8)}+Ñ_{(SCC)}+)+Ñ_{(CA15-3)}+Ñ_{(alpha-synuclein)}, Ñ_{NP}+Ñ_{IL-6}+Ñ_{(matrix metalloproteinase-8)}+Ñ_{(SCC)}+)+Ñ_{(CA15-3)}+Ñ_{(protein t-TAU)}, Ñ_{NP}+Ñ_{IL-6}+Ñ_{(matrix metalloproteinase-8)}+Ñ_{(SCC)+)}+Ñ_{(CA15-3)}+Ñ_{(colony stimulating factor 3)}, Ñ_{NP}+Ñ_{IL-6}+Ñ_{(matrix metalloproteinase-8)}+Ñ_{(SCC)+)}+Ñ_{(CA15-3)}+Ñ_{(C-X-C motif chemokine ligand 10)}, Ñ_{NP}+Ñ_{IL-6}+Ñ_{(matrix metalloproteinase-8)}+Ñ_{(SCC)+)}+Ñ_{(CA15-3)}+Ñ_{(C-C motif chemokine ligand 3)}, Ñ_{NP}+Ñ_{IL-6}+Ñ_{(matrix metalloproteinase-8)}+Ñ_{(SCC)+)}+Ñ_{(CA15-3)}+Ñ_{(C-C motif chemokine ligand 11)}, Ñ_{NP}+Ñ_{IL-6}+Ñ_{(CA125)}, +Ñ_{(TPS)}+Ñ_{(CA19-9)}+Ñ_{(CEA)}, Ñ_{NP}+Ñ_{IL-6}+Ñ_{(CA125)},+Ñ_{(TPS)}+Ñ_{(CA19-9)}+Ñ_{(CA15-3)}, Ñ_{NP}+Ñ_{IL-6}+Ñ_{(CA125)},+Ñ_{(SCC)}+Ñ_{(CA19-9)}+Ñ_{(CEA)}, Ñ_{NP}+Ñ_{IL-6}+Ñ_{(CA125)},+Ñ_{(SCC)}+Ñ_{(CA19-9)}+Ñ_{(tumor necrosis factor-alpha)}, Ñ_{NP}+Ñ_{IL-6}+Ñ_{(CA125)},+Ñ_{(SCC)}+Ñ_{(CA19-9)}+Ñ_{(interferon gamma)}, Ñ_{NP}+Ñ_{IL-6}+Ñ_{(matrix metalloproteinase-8)}+Ñ_{(SCC)}+Ñ_{(CA19-9)}+Ñ_{(interferon gamma)}, or Ñ_{NP}+Ñ_{IL-6}+Ñ_{(matrix metalloproteinase-8)}+Ñ_{(CEA)}+Ñ_{(CA15-3)}+Ñ_{(interferon gamma)}.

According to some embodiments, it may e.g. be advantageous to select the one or more additional biomarkers as disclosed herein according to the disease of said the human subject. For example, a predictive score S_{P} of the invention may reflect the clinical outcome/progression of a disease more reliably if the one or more additional biomarkers used in the calculation of the predictive score have been clinically associated with the respective disease. For example, the predictive score of the invention may more reliably predict the outcome or clinical progression of a human subject afflicted with lung cancer (e.g. NSCLC) if the one or more additional biomarkers are selected from a group of salivary biomarkers which have been shown to be associated with lung cancer such as interleukin receptor antagonist, interleukin-1 beta, interleukin-7, interleukin-8, interleukin-10, interleukin-13, colony stimulating factor 3, C-X-C motif chemokine ligand 10 (CXCL10), C-C motif chemokine ligand 3 (CCL3), C-C motif chemokine ligand 11 (CCL11), interferon-gamma, tumor necrosis factor-alpha. Accordingly, the predictive score may be calculated using Ñ_{NP}+Ñ_{IL-6}+Ñ of one, two, three, four, five or more of the above biomarkers.

According to one embodiment, it may e.g. be advantageous for human subjects afflicted with neurodegenerative diseases to use one or more biomarkers selected from the group of alpha-synuclein, amyloid beta 1-42, or protein t-TAU to calculate the predictive score according to the invention, e.g. Ñ_{NP}+Ñ_{IL-6}+Ñ_{(protein t-TAU)}, Ñ_{NP}+Ñ_{IL-6}+Ñ_{(amyloid beta 1-42)}, Ñ_{NP}+Ñ_{IL-6}+Ñ_{(alpha-synuclein)}, or Ñ_{NP}+Ñ_{IL-6}+Ñ_{(protein t-TAU)}+Ñ_{(amyloid beta 1-42)}, Ñ_{NP}+Ñ_{IL-6}+Ñ_{(protein t-TAU)}+Ñ_{(alpha-synuclein)}, Ñ_{NP}+Ñ_{IL-6}+Ñ_{(alpha-synuclein)}+Ñ_{(amyloid beta 1-42)}, or Ñ_{NP}+Ñ_{IL-6}+Ñ_{(protein t-TAU)}+Ñ_{(amyloid beta 1-42)}+Ñ_{(alpha-synuclein)}.

According to one embodiment, may e.g. be advantageous to use one or more biomarkers selected from the group of CA125, TPS, CA19-9, CEA, SCC (squamous cell carcinoma antigen) to calculate the predictive score of the invention if the human subject is afflicted with cancer or a neoplastic disease, e.g. Ñ_{NP}+Ñ_{IL-6}+Ñ_{(CA125)}, Ñ_{NP}+Ñ_{IL-6}+Ñ_{(TPS)}, Ñ_{NP}+Ñ_{IL-6}+Ñ_{(CA19-9)}, Ñ_{NP}+Ñ_{IL-6}+Ñ_{(CEA)}, Ñ_{NP}+Ñ_{IL-6}+Ñ_{(CA15-3)}, Ñ_{NP}+Ñ_{IL-6}+Ñ_{(SCC)}, or e.g. Ñ_{NP}+Ñ_{IL-6}+Ñ_{(CA125)}+Ñ_{(TPS)}, Ñ_{NP}+Ñ_{IL-6}+Ñ_{(CA125)}+Ñ_{(CA19-9)}, Ñ_{NP}+Ñ_{IL-6}+Ñ_{(CA125)}+Ñ_{(CEA)}, Ñ_{NP}+Ñ_{IL-6}+Ñ_{(CA125)}+Ñ_{(CA15-3)}, Ñ_{NP}+Ñ_{IL-6}+Ñ_{(CA125)}+Ñ_{(SCC)}, or e.g. Ñ_{NP}+Ñ_{IL-6}+Ñ_{(CA125)}+Ñ_{(SCC)}+Ñ_{(TPS)}, Ñ_{NP}+Ñ_{IL-6}+Ñ_{(CA125)}+Ñ_{(SCC)}+Ñ_{(CEA)}, Ñ_{NP}+Ñ_{IL-6}+Ñ_{(CA125)}+Ñ_{(SCC)}+Ñ_{(CA19-9)}, Ñ_{NP}+Ñ_{IL-6}+Ñ_{(CA125)}+Ñ_{(SCC)}+Ñ_{(CA15-3)}, or e.g. Ñ_{NP}+Ñ_{IL-6}+Ñ_{(CA125)}+Ñ_{(SCC)}+Ñ_{(CA15- 3)}+(TPS)+Ñ_{(CA19-9)}.

According to one embodiment, the normalization values for said one or more biomarkers as disclosed above which are used to calculate the predictive score according to the invention are:
interleukin receptor antagonist: 2810 pg/ml, interleukin-1 beta: 128 pg/ml, interleukin-7: 8.29 pg/ml, interleukin-8: 323 pg/ml, interleukin-10: 3.26 pg/ml, interleukin-13: 0.70 pg/ml, colony stimulating factor 3: 23.3 pg/ml, C-X-C motif chemokine ligand 10 (CXCL10): 949 pg/ml, C-C motif chemokine ligand 3 (CCL3): 2.28 pg/milliliter, C-C motif chemokine ligand 11 (CCL11): 5.33 pg/ml, interferon-gamma: 28.8 pg/ml, tumor necrosis factor-alpha: 12.5 pg/ml, total alpha-synuclein: 314 pg/ml, amyloid beta 1-42: 21.1 pg/ml, protein t-TAU: 9.6 pg/ml. The concentration of said one or more biomarkers in a given saliva sample from a human subject for which the predictive score of the invention is to be calculated may e.g. be determined as disclosed in Koizumi T et al, J Int Med Research 46, 3570, 2018 which uses a passive drool method to collect saliva samples, in which saliva is allowed to pool in the mouth of a human subject which is then allowed to drool it into a tube. In this method, the saliva sample is e.g. subjected to a multiplex bead array assay (Bi-Plex Pro Human Cytokine Grp I Panel 27-Plex; Bio-Rad Laboratories) according to the manufacturer's instructions to determine selected biomarker concentrations. Alternative methods for determining the concentration of one or more biomarkers in a given saliva sample from a human subject according to the invention are reviewed in Schepici G et al, Brain Sci 10, 245, 2020 each of which may be used in the inventive method such as such as ELISA, or magnetic bead-based Luminex assays to determine the concentration of selected biomarkers in salivary samples. The methodology as disclosed by Nagler R et al. Clin Cancer Res 12, 3979, 2006 discloses the use of a microparticle enzyme-linked immunoassay to determine the concentrations of the biomarkers SCC, CEA, CA19-9 and CA125. According to one embodiment, the clinical condition according to the invention is a decrease in organ function. As used herein the term “decrease in organ function” refers to a reduction of the organ function when compared to the average organ function of a control group. For example, a decrease in organ function may be caused by a reduction in the reparative and regenerative potential of the tissues that make up the organ. This reduction manifests as a decreased physiological reserve in response to stress, which may be referred homeostenosis and a time-dependent failure of complex molecular mechanisms that cumulatively create disorder.

According to one embodiment, a decrease in organ function according to the invention may affect any organ system within a human subject causing a clinical condition. For example, a decrease in organ function may be a decrease in lung function, heart function (cardiovascular function), renal function, musculoskeletal function, endocrine function, gastrointestinal function, or neurological function. For example, a decrease in lung function may be assessed by multiple methods in the art such as spirometry testing, or by comparison of molecular markers or biomarkers with those of a control group. For example, molecular markers such as plasma fibrinogen, CRP, Interleukins IL-6, and IL-8, total bilirubin, serum amyloid protein (SAA), surfactant protein D, club cell secretory protein 16 (CCSP-16) and Matrix Metalloproteinases MMP-8 and MMP-9 may be used to assess a decrease in lung function in comparison to a control group (see e.g. Pulmonol. 2018; 24(4):250-259). A decrease in heart (cardiac) function may be assessed by altered ECG patterns, or molecular markers for cardiac myocyte strain (e.g. BNP, NT-proBNP, or MR-proANP), markers of cardiac myocyte remodeling (ST2, galectin-3, GDF-15), or e.g. markers of inflammatory processes (e.g. Fas (Apol), TNF-alpha, CRP, Pentraxin-3), For example, a decrease in renal function according to the invention may be assessed by blood tests measuring blood urea nitrogen (BUN) creatinine, and glomerular filtration rate (GFR), changes in renal blood flow, or a decline of renal mass below e.g. 400 g, 350 g, 300 g, 250 g.

A decrease in musculoskeletal function in a human subject may e.g. be assessed using a questionnaire over a prolonged period of time to monitor a decline or decrease in musculoskeletal function (e.g. a questionnaire as published in J Bone Joint Surg Am 1999 September; 81(9):1245-60). A decrease in endocrine function may be assessed by analyzing hormone levels overtime in comparison to a control group or e.g. by a relative decline over time. Hormones that may e.g. be assessed according to the invention include melatonin, testosterone (in men), estrogen (in women), cortisol, insulin, thyroid hormones, or melatonin. Hormone level testing may e.g. be done using commercially available ELISA kits. For example, a decrease in gastrointestinal function may be assessed by determining changes in the gastrointestinal microbiome as described in GeroScience (2019) 41:935-9, or by monitoring motility of the GI tract over time, whereby a decrease in motility over time indicates a decrease in gastrointestinal function. Preferably, methods that do not require invasive procedures such as colonoscopy, are preferred methods to assess gastrointestinal function, whereby such procedures do not form part of the invention.

A decrease in neurological function may e.g. be assessed using cognitive tests such as those disclosed in Ther Adv Neurol Disord. 2012 November; 5(6): 349-358. Additional methods to assess neurological function may e.g. include functional magnetic resonance imaging or functional MRI (fMRI) measures brain activity by detecting changes associated with blood flow.

In one embodiment, the clinical condition according to the invention is a medical complication. The term “medical complication” as used herein refers to an unfavorable result of a disease, health condition, or treatment. Medical complications may adversely affect the prognosis, or outcome, of a disease. The development of medical complications depends on a number of factors, including the degree of vulnerability, susceptibility, age, health status, and immune system condition. Medical complications according to the invention may e.g. comprise renal failure, increased frailty according to the CSHA Frailty Index (e.g. as published in Mitniski A B et al. Sci. World J. 1:323-336, 2001), thromboembolic complications, gastrointestinal complications, cardiac complications, neurologic complications, metabolic complications.

For example, thromboembolic complications may include venous thromboembolism, disseminated intravascular coagulation (DIC), deep vein thrombosis following general anesthesia. Gastrointestinal complications may include gastrointestinal bleeding, or diarrhea, constipation, Inflammatory bowel disease (IBD), motility disorders, cleroderma, colonic inertia, gallbladder dysmotility, tachygastria, or gastroparesis. Cardiac complications according to the invention may e.g. include atrial fibrillation, or thrombosis in the heart, myocardial infarction, phlebitis, or endocarditis. Neurological complications according to the invention include e.g. anxiety disorders, schizophrenia, paradoxical reaction to a drug, depression. Neurological complications of the invention may e.g. also include ischaemic stroke, intracerebral haemorrhage, or CNS vasculitis. Ischaemic stroke, intracerebral haemorrhage, or CNS vasculitis as disclosed above may e.g. occur subsequent to a viral infection, such as an infection with a member of the coronaviridae family of viruses, e.g. betacoronavirus, such as SARS-CoV, or SARS-CoV2.

Medical complications according to the invention may e.g. also comprise metabolic complications, such as diabetes mellitus, diabetic neuropathy, diabetic retinopathy, or e.g. a metabolic syndrome, which is characterized by at least one of increased blood pressure, high blood sugar levels, and/or high triglyceride levels in the blood of affected human subjects.

According to one embodiment, the decrease in organ function and/or the risk of medical complications is a function of the age of the human subject. For example, the higher the age of the human subject the higher the risk of a medical complication herein and/or decrease in organ function both as disclosed herein. For example, a human subject at risk for a decrease in organ function and/or the risk of medical complications may be between 50 years of age to about 70, 80, 90 years of age, or from about 55, 60, 65, 70 years of age to about 75, 85, 95, 97, 98, 99, 100 years of age, or from about 45, 46, 47, 48, 49, 50, 51, 52, 53, 54, 55, 56, 57, 58, 59, 60 years of age to about 65, 66, 67, 68, 69 years of age, or from about 75, 76, 77, 78, 79, 80 years of age to about 81, 82, 83, 84, 85 years of age. The term “function of the age of the human subject” as used herein refers to a linear or exponential change of one or more characteristic parameters of a given organ. The change may affect expression levels of one or more genes, the capacity to tolerate oxidative stress, nerve conduction velocity. For example, a decrease in lung function may be a linear function of the age of the human subject, wherein the lung function which may be assessed by determining forced expiratory volume in 1 second (FEV1), forced vital capacity (FVC) and peak expiratory flow rate (PEFR), may decrease between 0.1%, 0.25%, 0.3% to about 0.5%, 0.6% per year in a human subject compared to an initial lung function if the human subject is about 60 years of age or older.

According to one embodiment, the decrease in organ function and/or the risk of medical complications is causally linked to inflammatory disease, degenerative syndromes, malignant tumors, or viral infections of the human subject. The term “causally linked” shall refer to the fact that any of inflammatory disease, degenerative syndromes, malignant tumors, or viral infections can cause a decrease in organ function and/or a risk of medical complications as a result of an acute or chronic state of the respective organ caused by a disease, syndrome, or infection.

In one embodiment, inflammatory disease as used according to the invention refers to acute inflammatory disease, or chronic inflammatory disease. For example, acute inflammatory disease (acute inflammation) may be caused by an allergic reaction, frostbite, chemical irritants, infection (e.g. bacterial or viral infection), burn, trauma, cuts, or laceration. Acute inflammatory diseases according to the invention also e.g. include appendicitis, bursitis, colitis, cystitis, dermatitis, epididymitis, encephalitis, gingivitis, meningitis, myelitis, nephritis, neuritis, pancreatitis, pharyngitis, phlebitis, tendonitis, vasculitis, acne vulgaris, asthma, diverticulitis, glomerulonephritis, inflammatory bowel syndrome, pelvic inflammatory disease, pneumonia (viral and bacterial), otitis, mastocytosis, or mast cell activation syndrome.

Accordingly, in one embodiment, of the invention the decrease in organ function and/or the risk of medical complications is causally linked to one of appendicitis, bursitis, colitis, cystitis, dermatitis, epididymitis, encephalitis, gingivitis, meningitis, myelitis, nephritis, neuritis, pancreatitis, pharyngitis, phlebitis, tendonitis, vasculitis, acne vulgaris, asthma, diverticulitis, glomerulonephritis, inflammatory bowel syndrome, pelvic inflammatory disease, pneumonia (viral and bacterial), otitis, mastocytosis, or mast cell activation syndrome.

According to one embodiment, the decrease in organ function and/or the risk of medical complications according to the invention is caused by one of the acute inflammatory diseases as disclosed above, or by two or more (e.g. two, three, four) acute inflammatory diseases as disclosed above.

Chronic inflammatory disease according to the invention may be one of cardiovascular disease, rheumatoid arthritis, autoimmune disease, neurological disease, or cancer. The term “cardiovascular disease” as used herein refers to a large number of diseases, or class of diseases that involve the heart or blood vessels (arteries and veins). Cardiovascular disease includes, but is not limited to, atherosclerosis, acute coronary syndrome, coronary artery disease, heart failure, vascular stenosis, particularly aortic stenosis, stroke, myocardial infarction, aneurysm, angina, myocarditis, valve disease, coronary artery disease, dilated cardiomyopathy, endocarditis, hypertension, heart failure, hypertrophic cardiomyopathy, myocardial infarction, transient ischemic attack (TIA), and venous thromboembolism.

The term “rheumatoid arthritis” or “RA” as used herein refer to a recognized disease state that may be diagnosed according to the “2010 American Rheumatoid Association criteria” (see e.g. The Journal of Rheumatology 2012; 39:11) for the classification of rheumatoid arthritis, or any similar criteria. The term includes not only active and early rheumatoid arthritis, but also incipient rheumatoid arthritis, as defined below. Rheumatoid arthritis includes, for example, juvenile-onset RA, juvenile idiopathic arthritis (JIA), or juvenile RA (JRA).

Another manifestation of a chronic inflammatory disease is autoimmune disease. The term “autoimmune disease” as used according to the invention refers to a condition arising from an abnormal immune response to a functioning body part. An autoimmune disease according to the invention is one of Achalasia, Addison's disease, adult Still's disease, agammaglobulinemia, alopecia areata, amyloidosis, ankylosing spondylitis, Anti-GBM/Anti-TBM nephritis, antiphospholipid syndrome, autoimmune angioedema, autoimmune dysautonomia, autoimmune encephalomyelitis, autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, dermatitis herpetiformis, Dermatomyositis, Devic's disease (neuromyelitis optica, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritism, Juvenile diabetes (Type 1 diabetes), Juvenile myositis (JM), Kawasaki disease,Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, Ill, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

According to one embodiment, the decrease in organ function and/or the risk of medical complications of the human subject according to the invention is causally linked to a chronic inflammatory disease, wherein the chronic inflammatory disease is a neurological disorder. The term “neurological disease” which is used interchangeably with “neurological disorder” as used herein refers to a brain dysfunction and neurodegeneration resulting from such diseases or disorders, including, but not limited to; Alzheimer's Disease (AD), Parkinson's Disease (PD), Huntington's Disease (HD), motor neuron disease, amyotrophic lateral sclerosis (ALS), cerebral palsy, genetic syndromes, epilepsy, spinal cord injury, neurodevelopmental malformations, or brain dysplasias.

According to one embodiment, the decrease in organ function and/or the risk of medical complications is causally linked to a degenerative syndrome. The term “degenerative syndrome” refers to the result of a continuous process based on degenerative cell changes, affecting tissues or organs, which will increasingly deteriorate over time. Preferably, the degenerative syndrome according to the invention is a neurodegenerative syndrome. The term “neurodegenerative syndrome” or “neurodegenetrative disorder” both terms of which are used interchangeably and as used herein refer to any disease or disorder which is caused by an impaired neuronal outgrowth, by neuronal death due to apoptosis, intoxication or necrosis, or by impaired neuronal function due to dendritic or axonal defects. A neurodegenerative disorder according to the invention may be one of Alzheimer's disease, Amyotrophic lateral sclerosis(ALS, Lou Gehrig's disease), Cancers, Charcot-Marie-Tooth disease (CMT), Chronic traumatic encephalopathy, Cystic fibrosis, Some cytochrome c oxidase deficiencies (often the cause of degenerative Leigh syndrome), Ehlers-Danlos syndrome, Fibrodysplasia ossificans progressiva, Friedreich's ataxia, Frontotemporal dementia (FTD), Huntington's disease, Infantile neuroaxonal dystrophy, Keratoconus (KC), Keratoglobus, Leukodystrophies, Macular degeneration (AMD), Marfan's syndrome (MFS), mitochondrial myopathies, Mitochondrial DNA depletion syndrome, Multiple sclerosis (MS), Multiple system atrophy, Muscular dystrophies (MD), Neuronal ceroid lipofuscinosis, Niemann-Pick diseases, Osteoarthritis, Osteoporosis, Parkinson's disease, Pulmonary arterial hypertension, prion diseases (Creutzfeldt-Jakob disease, fatal familial insomnia), Progressive supranuclear palsy, Retinitis pigmentosa (RP), Rheumatoid arthritis, Sandhoff Disease, Spinal muscular atrophy (SMA, motor neuron disease), Subacute sclerosing panencephalitis, Tay-Sachs disease, Vascular dementia.

According to one embodiment, the degenerative syndrome is selected from the group comprising Alzheimer's disease, amyotrophic lateral sclerosis, Friedreich's ataxia, Huntington's disease, Lewy body disease, multiple sclerosis, Parkinson's disease, Spinal muscular atrophy, prion disease, Pick's disease.

According to one embodiment, the decrease in organ function and/or the risk of medical complications of the human subject according to the invention is causally linked to a chronic inflammatory disease, wherein the chronic inflammatory disease is cancer. The term “cancer”, as used according to the invention, refers to diseases in which abnormal cells divide without control and can invade nearby tissues, and includes, but is not restricted to, acute lymphoblastic leukemia, acute myelogenous leukemia, bladder cancer, bone sarcoma, breast cancer, cervical cancer, Chorioadenoma destruens, choriocarcinoma, gastric cancer, Hodgkin lymphoma, hydatidiform mole, lung cancer, malignant mesothelioma, mycosis fungoides (a type of cutaneous T-cell lymphoma), neuroblastoma, non-Hodgkin lymphoma, non-small cell lung cancer, osteosarcoma, ovarian cancer, small cell lung cancer, soft tissue sarcoma, squamous cell carcinoma of the head and neck, testicular cancer, thyroid cancer, transitional cell bladder cancer, or Wilms tumor.

In one embodiment, the decrease in organ function and/or the risk of medical complications of the human subject according to the invention is causally linked to a chronic inflammatory disease, wherein the chronic inflammatory disease is a malignant tumor. The term “tumor” as used herein refers to uncontrolled growth of cancer cells in solid tissue such as an organ, muscle, or bone. A malignant tumors is not self-limited in its growth and has thus the tendency to become progressively severe. Malignant tumors according to the invention include tumors of connective tissue, endothelium and mesothelium, blood, lymphoid cells, muscle, epithelial tissues, neural tissue.

According to one embodiment, the decrease in organ function and/or the risk of medical complications of the human subject is causally linked to a chronic inflammatory disease, wherein the chronic inflammatory disease is a viral infection or caused by a viral infection. The term “viral infection” refers to a situation in which the human body is invaded by pathogenic viruses, and infectious virus particles (virions) attach to and enter susceptible cells. A viral infection according to the invention which results in the decrease in organ function and/or the risk of medical complications of the human subject may e.g. be caused by a virus selected from the group of viral families comprising herpesviridae, papovaviridae, arenaviridae, adenoviridae, hepadnaviridae, caliciviridae, astroviridae, bunyaviridae, coronaviridae, flaviviridae, orthomyxoviridae, paramyxoviridae, picornaviridae, pneumoviridae, reoviridae, retroviridae, rhabdoviridae, togaviridae.

According to a preferred embodiment, the virus according to the invention is selected from the family of coronaviridae, more preferably the alpha-coronavirus, beta-coronavirus, gamma-coronavirus or delta-coronavirus, particularly preferably the virus is a beta-coronavirus.

According to a particularly preferred embodiment, the beta-coronavirus according to the invention is SARS-CoV, or SARS-CoV2, including its variants such as alpha (Pango linage B.1.1.7), beta (Pango linage B.1.351), gamma (Pango linage P.1), delta (Pango linage B.1.617.2), or omicron (Pango linage B.1.1.529), nomenclature according to WHO nomenclature of SARS-CoV-2 variants of concern (VOCs).

In one embodiment, one or more devices are used to perform the testing to determine the concentration of salivary neutrophils and salivary IL6 according to the invention. The device may be a suitable for point-of-care testing, or alternatively may be medical equipment for laboratory use.

According to a preferred embodiment, one or more devices according to the invention are used to perform a point-of care testing to determine at least the concentration of salivary neutrophils and salivary IL-6 according to the invention. The term “point-of-care testing” (“POCT”) refers to medical diagnostic testing at or near the point of care that is, at the time and place of patient care. Point-of-care devices may be used according to the invention to determine the concentration of salivary neutrophils and/or salivary IL-6 as disclosed herein. For example, POCT devices as described in Bioanalysis, 24 Sep. 2019, 11(19):1777-1785, or Int J Lab Hematol. 2016 December; 38(6):703-709 may be used according to the invention to determine the salivary IL-6 concentration. The POCT testing may also be used to determine the concentration of at least one or more additional biomarkers as disclosed herein.

According to a preferred embodiment, the POCT device for use in the inventive method as disclosed herein of determining the number of salivary neutrophils and salivary IL-6 may be a lateral flow device. Lateral flow devices are known in the art and have e.g. been disclosed in WO2013/061026 A1, EP0810436 A1, or U.S. Pat. No. 7,858,396 B2. A lateral flow device according to the invention may e.g. comprise at least one, two, or three lateral flow strips for detecting an analyte such as IL-6, leukocyte, and/or interleukin receptor antagonist, interleukin-1 beta, interleukin-7, interleukin-8, interleukin-10, interleukin-13, colony stimulating factor 3, C-X-C motif chemokine ligand 10 (CXCL10), C-C motif chemokine ligand 3 (CCL3), C-C motif chemokine ligand 11 (CCL11), interferon-gamma, tumor necrosis factor-alpha, total alpha-synuclein, amyloid beta 1-42, protein t-TAU, CA125, TPS, CA19-9, CEA, SCC. An exemplary lateral flow device for the detection of leukocyte esterase is disclosed in WO 2007/027234 A2 the disclosure of which is hereby incorporated in its entirety. The lateral flow device according to the invention may e.g. be used to determine the leukocyte esterase activity in a sample which may in turn be used to extrapolate the number of salivary neutrophils in said sample based on a reference samples with a defined number of salivary neutrophils.

According to one embodiment, the lateral flow device for use in the inventive method can comprises two separate lateral flow strips, each capable of detecting a different analyte for the simultaneous detection of at least two analytes in a sample, such as saliva collected from a patient or test subject.

For example, in one embodiment, the lateral flow device according to the invention may comprise a first lateral flow strip for the detection of IL-6 and a second lateral flow strip which is different from the first lateral flow strip for the detection of a second analyte selected from the group comprising interleukin receptor antagonist, interleukin-1 beta, interleukin-7, interleukin-8, interleukin-10, interleukin-13, colony stimulating factor 3, C-X-C motif chemokine ligand 10 (CXCL10), C-C motif chemokine ligand 3 (CCL3), C-C motif chemokine ligand 11 (CCL11), interferon-gamma, tumor necrosis factor-alpha, total alpha-synuclein, amyloid beta 1-42, protein t-TAU, CA125, TPS, CA19-9, CEA, SCC, or leukocyte esterase, preferably, the second analyte is selected from amyloid beta, t-Tau, interleukin-10, SCC, or leukocyte esterase. The term “analyte” as used herein refers to a molecule to be detected in a test sample such as saliva, whereby the molecule to be detected is a biomarker according to the invention as disclosed herein.

According to a more preferred embodiment, the lateral flow device for use according to the invention comprises a first lateral flow strip for the detection of IL-6 and a second lateral flow strip which is different from the first lateral flow strip for the detection of a second analyte, wherein the second analyte is leukocyte esterase.

The use of a lateral flow device comprising a first and a second lateral flow strip for the detection of a first and second analyte as disclosed above may be advantageous as it only requires the handling of one lateral flow device.

According to some embodiments, lateral flow devices for use in the inventive method may also comprise more than two, e.g. three, four, five, or six, lateral flow strips in case more than two analytes are to be detected in a sample.

For example, lateral flow devices for the detection of three analytes in a sample according to the invention may comprise lateral flow strips for the detection of:

| 1st analyte to be detected | 2nd analyte to be detected | 3rd analyte to be detected |
|---|---|---|
| by 1st lateral flow strip | by 2nd lateral flow strip | by 3rd lateral flow strip |
| IL-6 | interleukin-10 | interleukin receptor |
| | | antagonist, interleukin-1 |
| | | beta, interleukin-7, |
| | | interleukin-8, interleukin-13, |
| | | colony stimulating factor 3, |
| | | C-X-C motif chemokine |
| | | ligand 10 (CXCL10), C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | interleukin-1 beta | interleukin receptor |
| | | antagonist, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CA19-9, CEA, SCC, or |
| | | leukocyte esterase |
| IL-6 | interleukin-7 | interleukin receptor |
| | | antagonist, interleukin-1 |
| | | beta, interleukin-8, |
| | | interleukin-10, interleukin-13, |
| | | colony stimulating factor 3, |
| | | C-X-C motif chemokine |
| | | ligand 10 (CXCL10), C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | interleukin-8 | interleukin receptor |
| | | antagonist, interleukin-1 |
| | | beta, interleukin-7, |
| | | interleukin-10, interleukin-13, |
| | | colony stimulating factor 3, |
| | | C-X-C motif chemokine |
| | | ligand 10 (CXCL10), C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | interleukin-10 | interleukin receptor |
| | | antagonist, interleukin-1 |
| | | beta, interleukin-7, |
| | | interleukin-8, interleukin-13, |
| | | colony stimulating factor 3, |
| | | C-X-C motif chemokine |
| | | ligand 10 (CXCL10), C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | interleukin-13 | interleukin receptor |
| | | antagonist, interleukin-1 |
| | | beta, interleukin-7, |
| | | interleukin-8, interleukin-10, |
| | | colony stimulating factor 3, |
| | | C-X-C motif chemokine |
| | | ligand 10 (CXCL10), C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | colony stimulating factor 3 | interleukin receptor |
| | | antagonist, interleukin-1 |
| | | beta, interleukin-7, |
| | | interleukin-8, interleukin-10, |
| | | interleukin-13, C-X-C motif |
| | | chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CA19-9, CEA, SCC, or |
| | | leukocyte esterase |
| IL-6 | C-X-C motif chemokine | interleukin receptor |
| | ligand 10 (CXCL10) | antagonist, interleukin-1 |
| | | beta, interleukin-7, |
| | | interleukin-8, interleukin-10, |
| | | interleukin-13, colony |
| | | stimulating factor 3, C-C |
| | | motif chemokine ligand 3 |
| | | (CCL3), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | C-C motif chemokine ligand | interleukin receptor |
| | 3 (CCL3) | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 11 |
| | | (CCL11), interferon-gamma, |
| | | tumor necrosis factor-alpha, |
| | | total alpha-synuclein, |
| | | amyloid beta 1-42, protein t- |
| | | TAU, CA125, TPS, CA19-9, |
| | | CEA, SCC, or leukocyte |
| | | esterase |
| IL-6 | C-C motif chemokine ligand | interleukin receptor |
| | 11 (CCL11) | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | interferon-gamma, tumor |
| | | necrosis factor-alpha, total |
| | | alpha-synuclein, amyloid |
| | | beta 1-42, protein t-TAU, |
| | | CA125, TPS, CA19-9, CEA, |
| | | SCC, or leukocyte esterase |
| IL-6 | interferon-gamma | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), tumor necrosis |
| | | factor-alpha, total alpha- |
| | | synuclein, amyloid beta 1-42, |
| | | protein t-TAU, CA125, TPS, |
| | | CA19-9, CEA, SCC, or |
| | | leukocyte esterase |
| IL-6 | tumor necrosis factor-alpha | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, total alpha- |
| | | synuclein, amyloid beta 1-42, |
| | | protein t-TAU, CA125, TPS, |
| | | CA19-9, CEA, SCC, or |
| | | leukocyte esterase |
| IL-6 | total alpha-synuclein | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, , amyloid beta |
| | | 1-42, protein t-TAU, CA125, |
| | | TPS, CA19-9, CEA, SCC, or |
| | | leukocyte esterase |
| IL-6 | amyloid beta 1-42 | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, protein t-TAU, |
| | | CA125, TPS, CA19-9, CEA, |
| | | SCC, or leukocyte esterase |
| IL-6 | protein t-TAU | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | CA125, TPS, CA19-9, CEA, |
| | | SCC, or leukocyte esterase |
| IL-6 | CA125 | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, TPS, CA19-9, |
| | | CEA, SCC, or leukocyte |
| | | esterase |
| IL-6 | TPS | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, CA19- |
| | | 9, CEA, SCC, or leukocyte |
| | | esterase |
| IL-6 | CA19-9 | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CEA, SCC, or leukocyte |
| | | esterase |
| IL-6 | CEA | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CA19-9, SCC, or leukocyte |
| | | esterase |
| IL-6 | SCC | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CA19-9, CEA, or leukocyte |
| | | esterase |
| IL-6 | Leukocyte esterase | interleukin receptor |
| | | antagonist, interleukin-1 |
| | | beta, interleukin-7, |
| | | interleukin-8, interleukin-10, |
| | | interleukin-13, colony |
| | | stimulating factor 3, C-X-C |
| | | motif chemokine ligand 10 |
| | | (CXCL10), C-C motif |
| | | chemokine ligand 3 (CCL3), |
| | | C-C motif chemokine ligand |
| | | 11 (CCL11), interferon- |
| | | gamma, tumor necrosis |
| | | factor-alpha, total alpha- |
| | | synuclein, amyloid beta 1-42, |
| | | protein t-TAU, CA125, TPS, |
| | | CA19-9, CEA. |

According to one embodiment, the results obtained by the lateral flow device of the invention as disclosed herein may e.g. be quantified using a lateral flow device reader. Exemplary lateral flow assay readers are disclosed in WO2013/061026 A, or WO2018/229477 A1. The use of such lateral flow device readers is advantageous, because numerical analyte concentrations are be provided which can be normalized and subsequently used to calculate the predictive score (S_{P}) according to the invention as disclosed above. In one embodiment, the lateral flow assay reader comprises a computing unit or is connected to a computer to transfer the numerical analyte concentrations to the computer to calculate the predictive score (S_{P}) according to formula (I) as disclosed above. The term “connected to a computer” as used herein refers to a functional connection between the lateral flow device reader and a computer or computing device which enables the transfer of the numerical analyte concentration data to the computer or computing device. For example, functional connections between the lateral flow device reader and the computing device or computer may include a connection via Bluetooth®, Ethernet, LAN, such as wireless LAN, any wireless protocols.

In further embodiments, the detection of e.g. IL-6 and leukocyte esterase for the calculation of the predictive score (S_{P}) according to the invention can e.g. be performed by using a combination of lateral flow assay device and/or lateral flow assay device reader and a device for quantitatively determining the concentration of leukocyte esterase (LE) such as the device disclosed in RSC Adv., 2020, 10, 27042.

In alternative embodiments, leukocyte esterase concentration in the inventive method may e.g. be determined by an internally calibrated electrochemical continuous enzyme assay (ICECEA) using an electrochemical substrate as disclosed in Anal. Chem. 2017, 89, 14, 7781-7787. Since the leukocyte esterase activity in a given sample is directly proportional to the number of leukocytes in a sample, e.g. salivary neutrophils in a salivary sample. For example salivary neutrophils display an average esterolytic activity of 0.86 and 1.4 nU, whereby one activity unit (U) of LE catalyzed the hydrolysis of 1.0 pmol of TAPTA per minute in a pH 7.40 phosphate buffer saline solution containing 10% dimethyl sulfoxide (DMSO) at 21° C. Thus, the leukocyte activity measured can be extrapolated to the number of cells in the sample. Alternative electrochemical methods that may be employed in the inventive method to determine the number of leukocytes in a sample, such as a saliva sample, are disclosed in WO2017/165222 which discloses the use of 4-((tosyl-L-alanyl)oxy)phenyl tosyl-L-alaninate in an internally calibrated electrochemical continuous enzyme assay.

In some embodiments, the point-of-care device according to the invention may e.g. be used to calculate the predictive score (S_{P}), whereby the determination of the concentration of both salivary IL-6 and salivary neutrophils and optionally of one or more additional biomarkers is controlled by a microprocessor which is programmed to calculate the predictive score (S_{P}) of the invention as disclosed herein. It is preferred that the point-of-care device can be programmed to use the normalization values as disclosed herein (e.g. 10 pg/ml salivary IL-6 and 90 salivary neutrophils/μl saliva, or e.g. one or more normalization values of the one or more additional biomarkers as disclosed herein) for the calculation of the predictive score (S_{P}). Alternatively, the device may be programmed to use normalization values different from those of the present invention as disclosed herein for calculating the predictive score.

According to one embodiment, the device according to the invention as disclosed above comprises one or more microfluidic biochip platforms to detect and quantify the concentration of at least salivary IL-6 and/or salivary neutrophils and optionally of one or more additional biomarkers as disclosed herein. For example, the device according to the invention comprises two microfluidic biochip platforms to detect and quantify salivary IL-6 and salivary neutrophils. Corresponding microfluidic devices that may be used according to the invention may be those as described in Sci Rep 6, 29410 (2016), or Lab Chip, 2018, 18, 522-531.

In one embodiment, any such microfluidics-based POCT device as disclosed above may also be connected to a network as disclosed above using the POCT01, or POCT1a2 protocol for data transfer, or any compatible version of said transfer protocol.

In one embodiment, the present invention pertains to a method of treating a human subject characterized by a predictive score S_{P}>0 which has been determined to be at a higher risk of an adverse clinical and/or treatment outcome. The method of treatment may e.g. be a precautionary treatment prior to the worsening of the medical condition of said human subject, or the treatment may be chosen to treat the first clinical manifestations of an increasing disease severity or disease activity.

For example, in case of a viral infection with SARS-Cov2 the human subject with a predictive score S_{P}>0 according to the invention may be treated with SARS-CoV2 neutralizing antibodies bamlanivimab, or casirivimab in combination with imdevimab, or dexamethasone, or a combination of dexamethasone with bamlanivimab, or casirivimab in combination with imdevimab, or combinations comprising at least Anti-IL-6-antibodies Tociluzumab and/or Sarilumab, or Anti-IL-6-antibodies Tociluzumab and/or Sarilumab, or combinations comprising at least Anti-IL-6-antibodies Tociluzumab and/or Sarilumab with imdevimab and/or dexamethasone whereby the treatment comprises administering to said human subject a therapeutically effective amount of said antibodies as disclosed above alone, or in combination with dexamethasone. Depending on the disease severity, the method of treatment may comprise administering remdesivir (alone or in combination with dexamethasone), or nirmatrelvir (PF-07321332) alone or in combination with ritonavir. The above treatment options may also comprise the supply of supplementary oxygen to said human subject.

In one embodiment, the present invention pertains to bamlanivimab for use in the inventive method of treating a human subject characterized by a predictive score S_{P}>0 afflicted with a viral infection with SARS-Cov2.

In one embodiment, the present invention pertains to casirivimab in combination with imdevimab for use in the inventive method of treating a human subject characterized by a predictive score S_{P}>0 afflicted with a viral infection with SARS-Cov2.

In one embodiment, the present invention pertains to tociluzumab for use in the inventive method of treating a human subject characterized by a predictive score S_{P}>0 afflicted with a viral infection with SARS-Cov2.

In one embodiment, the present invention pertains to sarilumab for use in the inventive method of treating a human subject characterized by a predictive score S_{P}>0 afflicted with a viral infection with SARS-Cov2.

In one embodiment, the present invention pertains to sarilumab in combination with imdevimab for use in the inventive method of treating a human subject characterized by a predictive score S_{P}>0 afflicted with a viral infection with SARS-Cov2.

In one embodiment, the present invention pertains to remdesivir for use in the inventive method of treating a human subject characterized by a predictive score S_{P}>0 afflicted with a viral infection with SARS-Cov2.

In one embodiment, the present invention pertains to nirmatrelvir (PF-07321332) for use in the inventive method of treating a human subject characterized by a predictive score S_{P}>0 afflicted with a viral infection with SARS-Cov2.

In one embodiment, the present invention pertains to nirmatrelvir (PF-07321332) in combination with ritonavir for use in the inventive method of treating a human subject characterized by a predictive score S_{P}>0 afflicted with a viral infection with SARS-Cov2. The method of treatment according to the invention of a human subject afflicted with an autoimmune disease as disclosed herein and which is characterized by a predictive score S_{P}>0 may be treated with a therapeutically effective amount of abatcept, or belatacept. The treatment with abatacept or belatacept may e.g. be particularly useful in patients with a predictive score S_{P}>0 afflicted with Multiple sclerosis, type I diabetes, psoriatic arthritis, systemic lupus (SLE).

The present invention is not to be limited in scope by the specific embodiments described herein. Indeed, various modifications of the invention in addition to those described herein will become apparent to those skilled in the art from the foregoing description and accompanying figures and detailed Examples which illustrate, by way of example, the principles of the invention.

### EXAMPLES

Clinical examples of the application of the present predictive score using a saliva test system

### Example 1—Prediction of an Increase in Age Related Morbidity and Frailty

Two subjects, subject I aged 69y and subject II aged 67y, both subjects presenting with anidentical CSHA Frailty index (Mitnitski A B et al., Sci. World J. 1: 323-336, 2001) of 0.08, were tested:
Subject I presented with a saliva interleukin-6 concentration of 20 pg/ml and a saliva neutrophil concentration of 100 cells/μl, resulting in a predictive score (S_{P}) of 1.1
Subject II presented with a saliva interleukin-6 concentration of 5 pg/ml and a saliva neutrophilconcentration of 600 cells/μl, resulting in a predictive score (S_{P}) of 5.7.

Based on the evaluation of the predictive scores of human subject I and human subject II, human subject I exhibiting a predictive score of 1.1 had a lower risk of increase in frailty when compared to human subject II exhibiting a higher predictive score of 5.7.

Within 6 months from time of saliva testing, subject I experienced a stable health and stable age-related condition as measured by the CSHA frailty index; subject II progressed toward an increased frailty index of 0.09, which is also accompanied by a higher risk of mortality.

### Example 2—Prediction of Progression of Inflammatory Disease

Two patients, patient I aged 58 years and patient II aged 55 years, both presenting with arthritis with an identical Disease Activity Score <3 (DAS28, Wells G et al., Ann. Rheum. Dis. 68: 954-960, 2009) which indicates a low disease activity, were tested according to the invention:
Patient I presented with a salivary interleukin-6 concentration of 10 pg/ml and a saliva neutrophil concentration of 200 cells/μl, resulting in a predictive score of 1.2.
Patient II presented with a salivary interleukin-6 concentration of 60 pg/ml and a saliva neutrophil concentration of 40 cells/μl, resulting in a predictive score of 5.0. Based on the evaluation of the predictive scores of patient I and patient II, it could be predicted that subject I exhibiting a predictive score of 1.2 had a lower risk of increasing arthritis disease activity compared to patient II who was characterized by a higher predictive score of 5.0.

Within 12 months from time of saliva testing, patient I experienced a continuous low arthritis disease activity as evidenced by a stable DAS28 score. For patient II a progression of the arthritis disease activity according to DAS28 was documented which increased to 4.0 corresponding to intermediate disease activity.

### Example 3—Prediction of Progression of Degenerative Syndrome

Two patients, patient I aged 70 years and patient II aged 73 years, both patients presenting with an identical Mini Mental State Examination (MMSE, Folstein M F et al., J. Psychiatr. Res. 12: 189-198, 1975) score >24 equaling minimal cognitive impairment, were tested according to the invention:
Patient I presented with a saliva interleukin-6 concentration of 35 pg/ml and a saliva neutrophil concentration of 50 cells/μl, resulting in a predictive score of 2.5. Patient II presented with a saliva interleukin-6 concentration of 10 pg/ml and a saliva neutrophil concentration of 150 cells/μl, resulting in a predictive risk score of 0.7.

Based on the evaluation of the predictive scores according to the invention of patient I and patient II, it could be inferred that patient I with a predictive score of 2.5 had a higher risk of decrease of cognitive functions when compared to patient II exhibiting a lower predictive score of 0.7.

Within 12 months from time of saliva testing, patient I experienced a moderate decrease in cognitive function as measured by a reduced MMSE score of 18; patient II exhibited no significant change of cognitive function as documented by stable MMSE.

### Example 4—Prediction of Cancer Related Deterioration

Two patients, patient I aged 53 years and patient II aged 55 years, both patients presenting with kidney cancer status post tumor nephrectomy with an identical Motzer risk score (J Clin Oncol. 1999 August; 17(8):2530-40) and an identical TNM stage (T3 N1 M+ with one bone and two pulmonary lesions below 1 cm each as documented by CT scan), were tested according to the invention:
Subject I presented with a saliva Interleukin-6 concentration of 40 pg/ml and a saliva neutrophil concentration of 700 per microliter, resulting in a predictive risk score of 9.8 Subject II presented with a saliva Interleukin-6 concentration of 10 pg/ml and a saliva neutrophil concentration of 70 per microliter, resulting in a predictive risk score of 0.

Based on the evaluation of the risk scores of patient I and patient II, it could be predicted that patient I exhibiting a predictive score (S_{P}) of 9.8 and had a higher risk of cancer progression when compared to patient II exhibiting a predictive score (S_{P}) of 0.

Within 3 months from time of saliva testing, patient I experienced a disease progression with new documented liver metastases; patient II remained stable with no signs of disease progression.

### Example 5—Prediction of Viral Infection Related Complications

Two healthy subjects, subject I aged 58y and subject II aged 54y, both subjects presenting with an identical medical history with no major viral or bacterial infections during the last 12 months, were tested according to the invention:
Subject I presented with a saliva interleukin-6 concentration of 10 pg/ml and a saliva neutrophil concentration of 70 cells/μl, resulting in a predictive score (S_{P}) of 0.
Subject II presented with a saliva interleukin-6 concentration of 30 pg/ml and a saliva neutrophil concentration of 500 cells/μl, resulting in a predictive score (S_{P}) of 6.6.

Based on the comparison of the predictive scores of subject I and subject II, it could be deduced that subject I exhibiting characterized by a predictive score (S_{P}) of 0 had a lower risk of clinical complications if infected with SARS-CoV-2, compared to subject II that is characterized by a higher predictive score (S_{P}) of 6.6.

Within 6 weeks from time of saliva testing, both subject I and subject II exhibited a documented infection with SARS-CoV-2; subject I initially experienced very mild and rapidly transient flu-like symptoms, and fully recovered within 10 days after the onset of infection; subject II deteriorated from time of infection and required intensive care treatment due to the severe course of the COVID-19 infection for a prolonged period of 4 weeks, followed by a 3 months period of ongoing severe chronic fatigue.

### Example 6—Prediction of Progression of Neuro-Degenerative Syndrome

Two subjects, subject I aged 74 years and subject II aged 76 years, both subjects presenting with an identical Mini Mental State Examination (MMSE, Folstein M F et al., J. Psychiatr. Res. 12: 189-198, 1975) score of 21 equaling mild cognitive impairment, were tested for saliva concentrations of Interleukin-6, neutrophils, amyloid beta 1-42, and protein t-TAU:

Subject I presented with saliva concentrations of IL6=50 pg/ml, salivary neutrophil concentration of 600 cells/μl, amyloid beta 1-42: 14 pg/ml, and protein t-TAU 6 ng/I (notably normal salivary concentrations of the known predictive markers amyloid beta 1-42, and protein t-TAU), resulting in an overall predictive risk score of 9.7

Subject II presented with saliva concentrations of 8 pg/ml, 200 cells/μl, 20 pg/ml, and 9 ng/I for Interleukin-6, neutrophils, amyloid beta 1-42, and protein t-TAU respectively (notably normal salivary concentrations of the known predictive markers amyloid beta 1-42, and protein t-TAU), resulting in an overall predictive risk score of 0.1

Based on the evaluation of the risk scores of subject I and subject II, it could be predicted that subject I exhibiting a predictive score S_{P} of 9.7 had a higher risk of a decrease of cognitive functions when compared to subject II exhibiting a lower predictive risk score of 0.1

Within 9 months from time of saliva testing, subject I experienced a significant decrease in mental function as measured by a reduced MMSE score of 13; subject II exhibited no significant change of cognitive function as documented by stable MMSE

### Example 7—Prediction of Cancer Related Deterioration

Two patients, patient I aged 65y and patient II aged 62y, both patients presenting with early stage squamous cell lung cancer status post radical tumor resection (including mediastinal lymph node dissection) and post adjuvant medical therapy, with an identical TNM stage (T2b NO MO) with no known metastatic diseases, were tested for saliva concentrations of Interleukin-6, neutrophils, Interleukin-10, SCC, and CXCL10:

Subject I presented with saliva concentrations of 10 pg/ml, 80 cells/μl, 2.5 pg/ml, 100 ng/ml, and 600 pg/ml for Interleukin-6, neutrophils, Interleukin-10, SCC, and CXCL10, respectively (notably normal salivary concentrations of the known predictive markers Interleukin-10, SCC, and CXCL10), resulting in an overall predictive score S_{P} of 0.

Subject II presented with saliva concentrations of 80 pg/ml, 1100 cells/μl, 2.5 pg/ml, 100 ng/ml, and 600 pg/ml for Interleukin-6, neutrophils, Interleukin-10, SCC, and CXCL10, respectively (notably normal salivary concentrations of the known predictive markers Interleukin-10, SCC, and CXCL10), resulting in an overall predictive risk score of 18.2.

Based on the evaluation of the predictive scores of patient I and patient II, it could be predicted that patient II exhibiting a predictive score of S_{P}=18.2 had a higher risk of imminent cancer progression when compared to patient I exhibiting a low predictive score S_{P} of 0

Within 6 months from time of saliva testing, patient I remained stable with no signs of disease progression; patient II experienced a disease progression with new documented pulmonary metastases.

1-34. (canceled)

35: A method of predicting the clinical and/or treatment outcome in a human subject at risk of clinical condition comprising
(i) determining the salivary concentration of Interleukin-6 (IL-6) in a salivary sample of said human subject; and
(ii) determining the concentration of salivary neutrophils in said salivary sample of said human subject, wherein the method further comprises calculating a predictive score for the calculation of which the at least two biomarkers salivary concentration of IL-6 and concentration of salivary neutrophils are used and wherein the normalized salivary IL-6 value (Ñ_{IL-6}) and normalized salivary neutrophil value (Ñ_{NP}) are used in a Theta Heaviside function wherein the normalized salivary IL-6 value (Ñ_{IL}-6) and normalized salivary value (Ñ_{NP}) are determined according to a scoring algorithm, comprising the steps of
i) assigning a predictive IL-6 score Ñ_{IL-6}=0, if the salivary IL-6 concentration is lower than or equal to the normalization value of IL-6,
ii) calculating Ñ_{IL-6} as the ratio of salivary IL-6 concentration:IL-6 normalization value and wherein the numerical value of 1 is subtracted from the normalization result yielding Ñ_{IL-6}, for a salivary IL-6 concentration which is greater than the IL-6 normalization value,
iii) assigning a predictive neutrophil (NP) score Ñ_{NP}=0, if the salivary neutrophil concentration is lower than or equal to the normalization value for salivary neutrophils,
iv) calculating Ñ_{NP} as the ratio of salivary neutrophil concentration: salivary neutrophil normalization value and wherein the numerical value of 1 is subtracted from the normalization result yielding Ñ_{NP}, for a salivary neutrophil concentration which is greater than the IL-6 normalization value, and wherein
the predictive score (S_{P}) is calculated as SP=Ñ_{NP}+Ñ_{IL-6}.

36: The method according to claim 35, wherein the salivary normalization value for IL-6 is 10 pg/ml and wherein the salivary normalization value for neutrophils is 90 cells/μl.

37: The method according to claim 36, wherein a predictive score (S_{P}) of S_{P}=0 indicates a low risk of an adverse clinical and/or treatment outcome and a predictive score S_{P}>0 indicates an increased risk of an adverse clinical and/or treatment outcome.

38: The method according to claim 36, wherein the clinical and/or treatment outcome between two or more human subjects at risk of prospective clinical condition is determined, wherein the method comprises calculating a predictive score S_{P} for each human subject, wherein the subject with the lowest predictive score has the lowest risk of an adverse clinical and/or treatment outcome.

39: The method according to claim 35, wherein the predictive score is calculated using one or more additional biomarkers, wherein the predictive score SP is calculated as
S_{P}=Ñ_{NP}+Ñ_{IL-6}+Σᵢ₌₁ⁿÑ(i),
wherein Ñ(i), is the predictive score of the one or more additional biomarker, wherein n is from 1 to 20, 21, 22, 23, 24 25, and wherein the normalized value (Ñ(i)) is determined according to a scoring algorithm, comprising the steps of
i) assigning a predictive Ñ(i)=0, if the concentration of said biomarker in said salivary sample is lower than or equal to the normalization value of said biomarker,
ii) calculating Ñ(i) as the ratio of said salivary biomarker (i) concentration: biomarker(i) normalization value and wherein the numerical value of 1 is subtracted from the normalization result yielding Ñ(i), for a salivary biomarker(i) the concentration of which is greater than respective normalization value of said biomarker (i).

40: The method according to claim 39, wherein the one or more additional biomarker is selected from the group comprising interleukin receptor antagonist, interleukin-1 beta, interleukin-7, interleukin-8, interleukin-10, interleukin-13, colony stimulating factor 3, C-X-C motif chemokine ligand 10, C-C motif chemokine ligand 3, C-C motif chemokine ligand 11, interferon-gamma, tumor necrosis factor-alpha, alpha-synuclein, amyloid beta 1-42, protein t-TAU, matrix metalloproteinase-8, CA125, TPS, CA19-9, CEA, CA 15-3, or SCC.

41: The method according to claim 40, wherein the normalization values for said one or more biomarkers are
interleukin receptor antagonist: 2810 pg/ml,
interleukin-1 beta: 128 pg/ml,
interleukin-7: 8.29 pg/ml,
interleukin-8: 323 pg/ml,
interleukin-10: 3.26 pg/ml,
interleukin-13: 0.70 pg/ml,
colony stimulating factor 3: 23.3 pg/ml,
C-X-C motif chemokine ligand 10 (CXCL10): 949 pg/ml,
C-C motif chemokine ligand 3 (CCL3): 2.28 pg/ml,
C-C motif chemokine ligand 11 (CCL11): 5.33 pg/ml,
interferon-gamma: 28.8 pg/ml,
tumor necrosis factor-alpha: 12.5 pg/ml,
total alpha-synuclein: 314 pg/ml,
amyloid beta 1-42: 21.1 pg/ml,
protein t-TAU: 9.6 pg/ml,
CA125: 384 units/ml,
TPS: 110 units/ml,
CA19-9: 27.1 units/ml,
CEA: 197.6 ng/ml,
SCC 140 ng/ml.

42: The method according to claim 35, wherein the clinical condition is a decrease in organ function, wherein the decrease in organ function is one of lung function, heart function, renal function, cardiovascular function, musculoskeletal function, endocrine function, gastrointestinal function, or neurological function.

43. The method according to claim 35, wherein the clinical condition is medical complication, wherein the medical complication is one of renal failure, increased frailty according to the CSHA Frailty Index, Thromboembolic complications, gastrointestinal complications, cardiac complications, neurologic complications, metabolic complications.

44: The method according to claim 42, wherein the decrease in organ function is a function of the age of the human subject.

45: The method according to claim 43, wherein the risk of medical complications is a function of the age of the human subject.

46: The method according to claim 42, wherein the prospective decrease in organ function is causally linked to inflammatory disease, degenerative syndromes, malignant tumors, or viral infections of the human subject.

47: The method according to claim 43, wherein the risk of medical complications is causally linked to inflammatory disease, degenerative syndromes, malignant tumors, or viral infections of the human subject.

48: The method according to claim 47, wherein the inflammatory disease is an acute inflammatory disease, or a chronic inflammatory disease.

49: The method according to claim 47, wherein the acute inflammatory disease is caused by an allergic reaction, frostbite, chemical irritants, infection, burn, trauma, cuts, or laceration.

50: The method according to claim 47, wherein the chronic inflammatory disease is cardiovascular disease, rheumatoid arthritis, autoimmune disease, neurological disease, or cancer.

51: The method according to claim 47, wherein the degenerative syndrome is selected from the group comprising Alzheimer's disease, amyotrophic lateral sclerosis, Friedreich's ataxia, Huntington's disease, Lewy body disease, multiple sclerosis, Parkinson's disease, Spinal muscular atrophy, prion disease, Pick's disease.

52: The method according to claim 47, wherein the malignant tumor is a malignant tumor of connective tissue, endothelium and mesothelium, blood, lymphoid cells, muscle, epithelial tissues, neural tissue.

53: The method according to claim 47, wherein the viral infection is caused by a virus selected from the group viral families comprising herpesviridae, papovaviridae, arenaviridae, astroviridae, bunyaviridae, coronaviridae, flaviviridae, orthomyxoviridae, paramyxoviridae, picornaviridae, reoviridae, retroviridae, rhabdoviridae, togaviridae.

54: The method according to claim 35, wherein one or more devices are used to perform point-of-care testing (POCT devices) to determine at least the concentration of salivary neutrophils and/or salivary IL-6.

55: The method according to claim 54, wherein the one or more POCT devices used comprise one or more microfluidic biochip platforms to detect and quantify salivary IL-6 concentration and/or the salivary neutrophil concentration.

56: The method according to claim 54, wherein the POCT device is a lateral flow device, wherein the lateral flow device comprises at least two lateral flow strips for the quantification of IL-6 and a second biomarker selected from the group comprising interleukin receptor antagonist, interleukin-1 beta, interleukin-7, interleukin-8, interleukin-10, interleukin-13, colony stimulating factor 3, C-X-C motif chemokine ligand 10 (CXCL10), C-C motif chemokine ligand 3 (CCL3), C-C motif chemokine ligand 11 (CCL11), interferon-gamma, tumor necrosis factor-alpha, total alpha-synuclein, amyloid beta 1-42, protein t-TAU, CA125, TPS, CA19-9, CEA, SCC, or leukocyte esterase.

57: A method of treating a human subject characterized by predictive score S_{P}>0 indicating that the human subject has an increased risk of an adverse clinical and/or treatment outcome, wherein predictive score is calculated using a Theta Heaviside function using at least a normalized salivary IL-6 value (Ñ_{IL-6}) and a normalized salivary neutrophil value (Ñ_{NP}), wherein the normalized salivary IL-6 value (Ñ_{IL-6}) and normalized salivary value (Ñ_{NP}) are determined according to a scoring algorithm, comprising the steps of
a) assigning a predictive IL-6 score Ñ_{IL-6}=0, if the salivary IL-6 concentration is lower than or equal to the normalization value of IL-6,
b) calculating Ñ_{IL-6} as the ratio of salivary IL-6 concentration:IL-6 normalization value and wherein the numerical value of 1 is subtracted from the normalization result yielding Ñ_{IL-6}, for a salivary IL-6 concentration which is greater than the IL-6 normalization value,
c) assigning a predictive neutrophil (NP) score Ñ_{NP}=0, if the salivary neutrophil concentration is lower than or equal to the normalization value for salivary neutrophils,
d) calculating Ñ_{NP} as the ratio of salivary neutrophil concentration: salivary neutrophil normalization value and wherein the numerical value of 1 is subtracted from the normalization result yielding Ñ_{NP}, for a salivary neutrophil concentration which is greater than the IL-6 normalization value, and wherein the predictive score (S_{P}) is calculated as S_{P}=Ñ_{NP}+Ñ_{IL-6}.

58: The method of treating a human subject according to claim 57, wherein the treatment is a precautionary treatment prior to the worsening of the medical condition, or wherein the treatment is chosen to treat the first clinical manifestations of an increasing disease severity or activity.
